(19) United States
(12) Patent Application Publication
(10) Pub. No.: US 2022/0203051 A1
(43) Pub. Date: Jun. 30, 2022

(54) Medix/Boost Nasal inhaler

(71) Applicant: Sol Gersh, Bal Harbour, FL (US)

(72) Inventor: Sol Gersh, Bal Harbour, FL (US)

(21) Appl. No.: 16/974,329

(22) Filed: Dec. 31, 2020

(51) Int. Cl. A61M 15/08; A24F 40/10; A24B 15/167; A24F 7/04; A24D 3/17; A24B 15/32; A24B 15/30; A24B 15/38; A61K 38/06; A61K 31/05; A61K 36/45; A61K 36/61; A61K 36/48; A61K 31/4745; A61M 15/00

(52) U.S. Cl. CPC A61M 15/08; A24F 40/44; A24B 15/167; A24F 7/04; A24D 3/17; A24B 15/32; A24B 15/303; A24B 15/385; A61K 38/063; A61K 31/05; A61K 36/45; A61K 36/61; A61K 36/48; A61K 31/4745; A61M 15/0021; A24F 40/10

(57) ABSTRACT

Combined smoking/vaporizing articles for inhaling vapor. Contains an extra

Compartment for a replaceable triple filter with a unique main ingredient that

Has special properties: A.) kills bad bacteria in the body B.) strong Anti-inflammatory C.) BOOSTS the immune system , helping to fight all major diseases, including Covid-19.

### FIELD OF THE INVENTION

The present invention relates generally to an apparatus for an electronic cigarette. More specifically, the present invention is an apparatus for an electronic cigarette with an extra compartment for the replaceable filter.

### BACKGROUND OF THE INVENTION

The present invention is an electronic cigarette with an extra compartment for the replaceable filter (recommended once a week) cleaner, safer, and healthier.

### BRIEF DESCRIPTION OF THE DRAWINGS

FIG. 1 is a drawing of the present invention;

FIG. 2 is a drawing of the present invention;

FIG. 3 is a drawing of the present invention.

### DETAIL DESCRIPTIONS OF THE INVENTION

All illustrations of the drawings are for the purpose of describing selected versions of the present invention and are not intended to limit the scope of the present invention.

The present invention is an electronic cigarette with an extra compartment for the replaceable filter (recommended once a week) cleaner, safer, and healthier. The present invention is for construction of an electronic cigarette with the nicotine(+flavor) at one end, the mouthpiece at the other end, and the additional compartment to receive a replaceable filter in between.

There are 10 different applications of this triple technology for various filter in specific shapes to clean water, air, and pollution.

Autos; air conditioners; industrial (factories); bottled water; room air fresheners; refrigerators; hot tubs; and swimming pools, nasal inhalers; respirators; coffee; cigar and cigarette holders. Also, with an area for the replaceable medix filter. Medix triple filter can come in various sizes and shapes; same basic formula: mesh filter at one end, medix mixture in middle, and charcoal at end.

The present invention is based upon the previous provisional patent “new construction of class 034 smoking items; cigarettes, e-cigarettes, and holders.

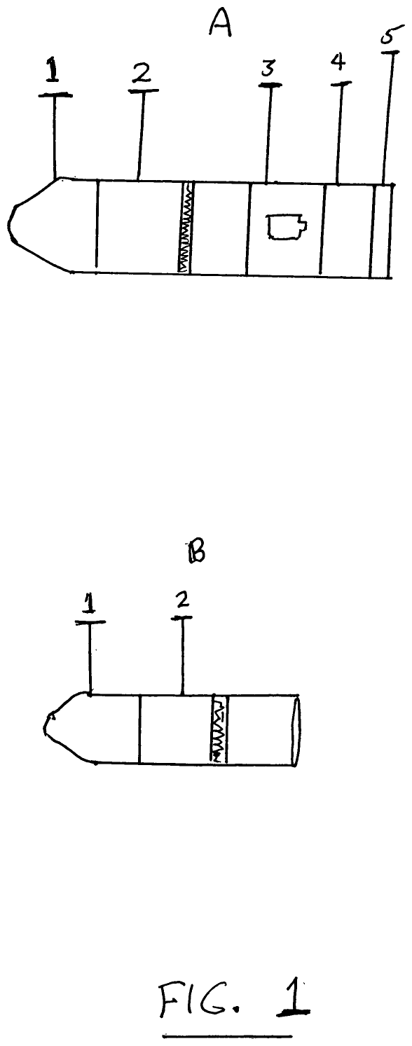

This is not a design patent as the exact size and shape are not important. It is the idea and the construction of an e-cigarette (as shown in FIG. 1). The present invention comes with an additional compartment located between the mouthpiece and the other elements to house a replaceable (medix triple) filter.

The present inventor has visited several smoke shops can called several other and no one has such an item nor do they know of any to exist. By adding additional filtration (and the medical benefits of medic plants and the special antioxidant) this creates a cleaner, safer smoking experience (replace filter about once a week).

The fabaceous plants in the medic-mixture have been used for thousands of years in ancient Egypt, India, China, etc, by boiling in water and inhaling the vapor to treat sore throats, coughs, colds, flu, asthma, bronchitis, and sinus inflammation with no side effects. They are still in use today in 80% of the countries in the world. The antioxidant is present in every human being without which they cannot live. We start to lose the ability to produce it at age 45. It kills bad bacteria in the body and boosts the immune system helping to fight against all major diseases; cancer, stroke, heart attack, etc and is a treatment by inhaling for C.O.P.D, the 4^{th} largest global cause of death. The problem solved it that this makes smoking safer and more beneficial for health.

Version of invention equals 2 standard filets with an additional center compartment with the medic mixture.

Relationship between components; 90% of all cigarettes are filter tip to add protection and safety. The medix triple filter adds time tested medical benefits such as antibacterial, antigerm, antifungal, antivirus, and anti inflammatory. Whether the medix filter is added to a cigarette, e-cigarette, cigar, or cigarette holder, there are added benefits.

How to use the present invention; there are 1.1 billion smokers and growing, it is important to make smoking as safe as possible.

When using the medix e-cigarette without nicotine, the user has an instrument that can deliver soothing vapors with numerous medical benefits to treat several ailments because the medic plants and the antioxidant are in the filter. The present inventors father died at age 65 from smoking, the legacy will be to help people live healthier and longer.

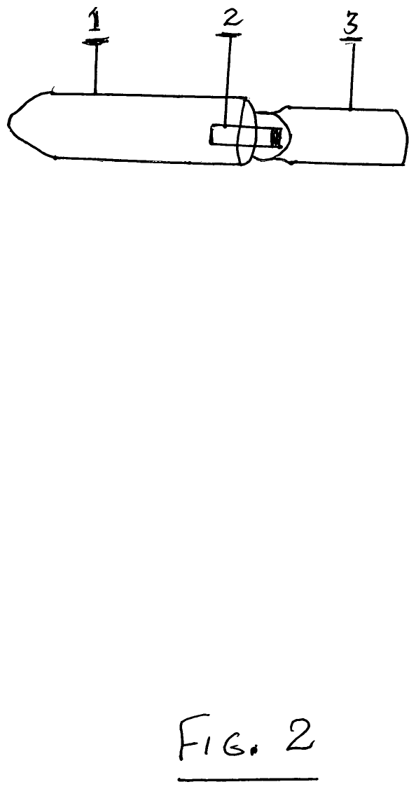

Configuration for production for two e-cigarettes; (shown in FIG. 2) metal with (B) section containing the nicotine compartment, heating element, and battery which screws into (A) mouthpiece which contains a compartment to house the replaceable (medix) filter.

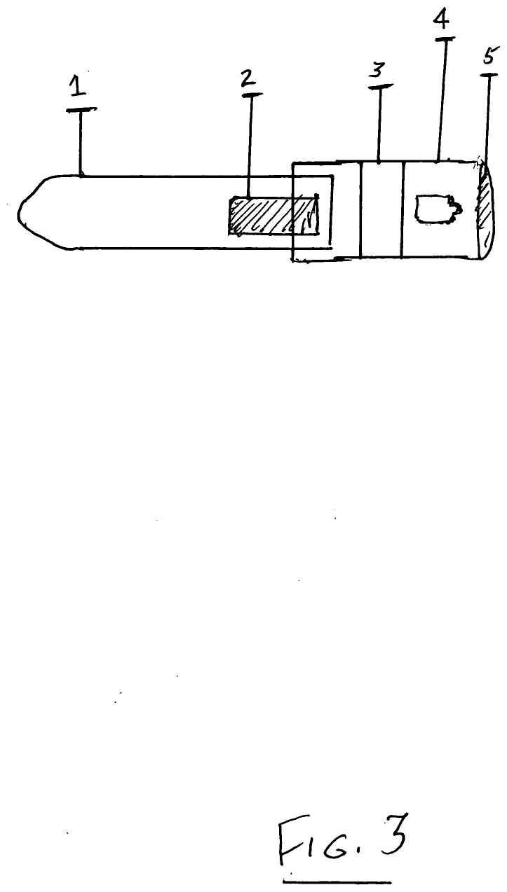

Shown in FIG. 3, plastic, which contains in part (B) nicotine compartment, heating element, and battery and which slides over. Mouthpiece (A) which contains a compartment to house the replaceable filter.

Although the invention has been explained in relation to its preferred embodiment, it is to be understood that many other possible modifications and variations can be made without departing from the spirit and scope of the invention.

### BRIEF DESCRIPTION OF THE DRAWINGS

FIG. 1A is a drawing of the present invention. Cross Sectional view. is the mouthpiece. 2 triple filter replace 3 battery 4 heating coil 5 nicotine Medical instrument or Electronic cigarette B is a cigarette holder 1 mouthpiece 2 triple filter replaceable

FIG. 2 drawing of the present invention. Cross sectional view 1 mouthpiece 2 triple filter replaceable 3 metal end screw into 1 Medical instrument or Electronic cigarette.

FIG. 3 is a drawing of present invention. Cross sectional view plastic Construction. 1 mouthpiece 2 filter replaceable 3 coil 4 battery 5 nicotine Medical instrument or Electronic cigarette

Medix Special Function

The Medix/boost nasal inhaler, as is, and the Medix healthy E-cigarette When used without nicotine; act like medical instruments: with benefits: Anti bacteria, virus, fungus, germs, and anti inflammatory. Glutathione, The main ingredient is edible , but is documented to be most effective BY INHALING THE VAPOR! These ingredients have been used for thousands Of years without any side effects. 80% of all countries in the world, now Employ this same method to treat respiratory diseases: INHALING THE VAPOR Glutathione BOOSTS the immune system!.

1. (canceled)

2. (canceled)

3. (canceled)

4. (canceled)

5. (canceled)

6. A nasal inhaler apparatus comprising a tubular hollow compartment, a mouthpiece, and a replaceable triple filter consisting of a mesh filter at one end. a charcoal filter at the other end, and a mixture in the middle, wherein the mixture contains effective amounts of glutathione, pyrroloquinoline quinione, alfalfa, menthol oil, eucalyptus oil, and wintergreen oil.
